SEQUENCE LISTING (1 sequence)

SEQ ID NO: 1 - DNA, 18 bp, unknown
  nonpalindromic nucleotide
tagggataacagggtaat

(19) United States
(12) Patent Application Publication
(10) Pub. No.: US 2003/0092161 A1
(43) Pub. Date: May 15, 2003

(54) Compositions and methods for production of recombinant viruses, and uses therefor

(75) Inventors: Guangping Gao, Rosemont, PA (US); James M. Wilson, Gladwyne, PA (US); Mauricio R. Alvira, Philadelphia, PA (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(73) Assignee: The Trustees of the University of Pennsylvania, Philadelphia, PA (US)

(21) Appl. No.: 10/246,447

(22) Filed: Sep. 19, 2002

Related U.S. Application Data
(60) Provisional application No. 60/323,352, filed on Sep. 19, 2001.

(51) Int. Cl. C12N 7/00; C12N 7/02; C12N 15/86

(52) U.S. Cl. 435/235.1; 435/239; 435/456

(57) ABSTRACT

An efficient method of producing recombinant virus is described. The method involves transfecting an uncut, circular plasmid containing a recombinant viral genome, a first I-SceI recognition site located 5′ to the viral genome, and a second I-SceI recognition site located 3′ to the adenovirus genome into a host cell. The host cell is then cultured under conditions in which it expresses I-SceI endonuclease. The endonuclease cleaves the I-SceI recognition sites, thereby rescuing the recombinant virus from the plasmid, making it available for packaging into an infectious virus.

### CROSS-REFERENCE TO RELATED APPLICATIONS

This application claims the benefit of the priority of U.S. Patent Application No. 60/323,352, filed on Sep. 19, 2001.

### BACKGROUND OF THE INVENTION

Obtaining the DNA sequence of the entire human genome only heralds the end of the beginning of the human genome project. The next step is ‘functional genomics’, i.e., developing an understanding of the functions of the decoded human genes and elucidating the organization and control of different gene pathways that, put together, make up the human physiology. In order to facilitate the study of functional genomics, it is necessary to find tools which will efficiently deliver genes to animal models or target cells and which will achieve high level expression of these genes to permit study of their biological roles in the recipients. One attractive tool for functional genomics studies is an adenovirus-based viral vector because it is easily constructed, propagated and purified, has a wide host range, and is able to transduce dividing and quiescent cells in vitro and in vivo efficiently.

Traditionally, recombinant adenoviruses have been generated through homologous recombination by cloning the gene of interest into a shuttle plasmid and then co-transfecting the shuttle plasmid with restricted viral backbone DNAs into the human kidney 293 cells or other required complementing cells lines. This process usually leads to a mixture of recombinant viruses and requires a lengthy plaque purification process to isolate relatively uniform species of viruses

There is a need to overcome the limitations in adenoviral production methods.

### SUMMARY OF THE INVENTION

In one aspect, the invention provides a method of rescuing recombinant virus. The method involves transfecting into a host cell an uncut, circular plasmid containing a recombinant viral genome, a first rare restriction enzyme recognition site located 5′ to the viral genome, and a second rare restriction enzyme recognition site located 3′ to the viral genome. Suitably, the host cell is stably transformed with the corresponding rare restriction enzyme(s), or optionally, the host cell may be provided with the enzyme in trans. The host cells are then cultured under conditions in which the rare restriction enzyme cleaves the recognition sites, thereby rescuing the intact recombinant adenovirus genome from the plasmid and making it available for encapsidation into an infectious viral particle.

In another aspect, the invention provides a host cell containing an uncut, circular plasmid containing a recombinant viral genome, a first rare restriction enzyme recognition site located 5′ to the viral genome, and a second rare restriction enzyme recognition site located 3′ to the viral genome. In another embodiment, the host cell further contains a nucleic acid molecule comprising nucleic acid sequences encoding the rare restriction enzyme in the host cell.

In a further aspect, the invention provides a method of producing recombinant virus by transfecting a host cell stably transformed with the rare restriction enzyme with an uncut, circular plasmid as described herein.

These and other advantages of the invention will be readily apparent from the following detailed description of the invention.

### DETAILED DESCRIPTION OF THE INVENTION

The invention provides a rapid method for producing recombinant viruses. The method involves transfecting into a host cell an uncut, circular plasmid containing a recombinant adenovirus genome, a first rare restriction enzyme site located 5′ to the adenovirus genome, and a second rare restriction enzyme site located 3′ to the adenovirus genome. Suitably, the host cell is stably transformed with the enzyme corresponding to the rare site (e.g., I-SceI endonuclease for I-SceI sites), or optionally, the host cell may be provided with the enzyme in trans. The host cells are then cultured under conditions in which the enzyme cleaves the recognition sites, thereby rescuing the recombinant adenovirus from the plasmid.

Advantageously, the method of the invention avoids the extra step required by prior art methods which require linearization of the plasmids containing cloned viral genomes prior to transfection. By avoiding linearization and permitting cleavage of the intact recombinant viral genome, the method of the invention also avoids variations in the quality of DNAs caused by linearization using restriction enzyme sites which are present in multiple locations throughout the viral genome, reducing transfection efficiency.

The method of the invention is suited for use in production of any recombinant virus which requires packaging into a capsid or envelope to render it infectious. For example, the method may be utilized in production of alphavirus, adenovirus, adeno-associated virus, baculoviruses, delta virus, hepatitis viruses, herpes viruses, lentiviruses, filoviruses, pox viruses, papova viruses, poliovirus, pseudorabies viruses, parvoviruses, retroviruses, and vaccinia viruses, amongst others. The method may also be utilized in production of chimeric, pseudotyped, and other manipulated viruses. In one particularly desirable embodiment, the method is utilized for the production of recombinant adenoviruses. Although the examples provided herein make reference to adenoviruses, it will be readily understood by one of skill in the art that the method of the invention may be used for other recombinant viruses, as desired.

The methods of the invention, and the compositions useful for this method, including plasmids, host cells and the like, are described in further detail as follows.

I. Rare Restriction Enzymes Recognition Sites and their Corresponding Enzymes

As used herein, the term “rare-cutting restriction enzyme (recognition) site” refers to the sequence of nucleotides (i.e., “site”) cleaved by a restriction enzyme which site is located infrequently (e.g., 1 to 3 copies), or more preferably, is completely absent from the recombinant viral genome. Most desirably, the rare restriction enzyme site is also rare or absent from the remainder of the plasmid. Suitable rare-cutting restriction enzymes sites are at least about 12 to about 40 nucleotides in length, preferably about 14 to about 20 nucleotides in length, and most preferably, at least about 18 nucleotides in length. Based on the information provided herein, one of skill in the art can readily select a suitable rare-cutting restriction enzyme site.

Suitable rare-cutting restriction enzymes that recognize such sites may be selected, for example, from among various restriction enzymes which are native to non-mammalian animals, plants, yeast, fungi, and/or insects, which restriction enzymes are not native to mammalian species. Examples of rare-cutting restriction enzymes, include, in addition to I-SceI, PspI and I-CeuI.

In the examples provided herein, the rare restriction endonuclease I-Sce-I is utilized. The I-SceI enzyme is an endonuclease encoded by the group I intron of S. cerevisiae mitochondria [L. Colleaux et al, Proc. Natl. Acad. Sci. USA, 85:6022-6026 (1988), which has high specificity for an 18 bp nonpalindromic nucleotide sequence [I-SceI site: SEQ ID NO: 1: 5′-tagggataa/cagggtaat]. In a human genome with about 3×10⁹ nucleotides, a common restriction endonuclease generally recognizes a short stretch of nucleotides of 4 to 8 base pairs; thus, there would be about one million such sites in one human genome. In contrast, the I-SceI site occurs randomly only once in every 20 human genomes. The rarity of I-SceI sites has been partially confirmed by the fact that there are no I-SceI sites in the genomes of many organisms, including viruses, bacteria and yeast.

Thus, rare-cutting restriction enzymes are selected based upon the frequency of occurrence of the sites which they recognize. Such restriction enzymes are available from a variety of commercial sources, including, e.g., New England BioLabs, Promega, and Boehringer Mannheim. Alternatively, these enzymes or their coding sequences may be produced synthetically or using recombinant technology. For example, the I-SceI enzyme may be purchased from commercial sources (e.g., Boehringer Mannheim, Germany). Alternatively, the sequence of the enzyme may be produced by conventional chemical synthesis. See, e.g., Barony and Merrifield, cited below. Preferably, the native coding sequence for this enzyme (or another selected rare-cutting restriction enzyme) is altered to optimize expression in mammalian cells, which are the preferred host cells. Techniques for optimizing expression, e.g., by altering preference codons, are well known to those of skill in the art.

The sequences for rare-cutting restriction enzyme recognition sites and the enzymes which recognize these sites, may be produced synthetically, recombinantly, or obtained using other suitable techniques. See, e.g., G. Barony and R. B. Merrifield, The Peptides: Analysis, Synthesis & Biology, Academic Press, pp. 3-285 (1980)]; Sambrook et al, Molecular Cloning: A Laboratory Manual, Cold Spring Harbor Laboratories, Cold Spring Harbor, N.Y.

The examples provided herein utilize the I-SceI recognition sites and its corresponding endonuclease and reference is made the I-SceI throughout the specification for convenience. However, it will be understood that one of skill in the art could utilize other rare enzyme sites (and their corresponding enzymes) as described herein.

II. Plasmid Carrying Viral Genome Flanked by Rare Restriction Enzyme Sites

As used herein, the term “plasmids” refers to any small, circular DNA molecule that replicates independently from the chromosome of the host cell. The plasmids useful in this invention may be engineered such that they are suitable for replication and, optionally, integration in prokaryotic cells, mammalian cells, or both.

In order to perform the method of the invention, an uncut plasmid carries, at a minimum, a selected recombinant viral genome which is flanked by one or more rare-cutting restriction enzyme recognition sites located at each the 5′ end and 3′ end of a recombinant viral genome. Optionally, the plasmid may contain a second expression cassette, containing sequences encoding the rare restriction enzyme and regulatory control sequences therefor. These and other components of the plasmid and viral genome are discussed below.

A. Rare Restriction Enzyme Sites

In a plasmid of the invention, the rare-cutting restriction enzyme recognition sites flank the viral genome, so that upon digestion with the corresponding enzyme, the intact viral genome is excised from the plasmid. Thus, the plasmid carrying the viral genome is engineered such that the rare restriction recognition sites are located 5′ and 3′ to the viral genome. In one desired embodiment, the recognition sites for a single rare-cutting restriction enzyme are used. However, in other embodiments, it may be desirable to utilize more than one rare restriction enzyme and the corresponding recognition sites therefor Suitably, the plasmid contains multiple copies of the rare restriction enzyme recognition sites flanking the viral genome to improve efficiency. For example, the plasmid may contain two, three, or more copies of the rare restriction enyzme recognition sites flanking the genome.

In one embodiment, the rare restriction enzyme recognition sites are located immediately 5′ and/or immediately 3′ to the viral genome. Alternatively, there may be a spacer between the 5′ end of the viral genome and the first rare restriction enzyme recognition site and/or between the 3′ end of the viral genome and the second rare restriction enzyme recognition site.

As used herein, a spacer may be any DNA sequence of about 1 to about 10 bases which is interposed between the end of the viral genome and the rare restriction enzyme site. The spacer may have any desired design; that is, it may be a random sequence of nucleotides, or alternatively, it may encode a gene product, such as a marker gene The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls.

In addition, the plasmid may be designed to include one or more rare restriction enzyme sites as defined herein. Such sites may be located within the spacer region, or more desirably, 5′ and/or 3′ to the rare restriction enzyme recognition sites.

B. The Recombinant Viral Genome

In one particularly desirable embodiment, the circular plasmid contains a recombinant viral genome located between the rare restriction enzyme sites The recombinant viral genome contains the minimal viral elements necessary to permit packaging of the viral genome in a capsid or envelope to produce an infectious virus and a minigene cassette for delivery to a host cell. Suitable sequences for the recombinant viral genome, including the minigene, may be readily obtained by one of skill in the art from a variety of public, commercial, and academic sources.

1. Viral Sequences

In one embodiment, the recombinant viral genome is a viral genome containing the minimal viral sequences necessary to enable a viral particle to be produced with the assistance of a helper virus and/or, optionally, a packaging cell line. One of skill in the art can readily select the necessary sequences depending upon the type of recombinant virus to be produced.

Where the virus is an adenovirus, the minimal adenoviral sequences necessary to include in the recombinant viral genome for replication and virion encapsidation into an infectious adenoviral particle are the cis-acting 5′ and 3′ inverted terminal repeat (ITR) sequences of an adenovirus. More particularly, the entire adenovirus 5′ sequence containing the 5′ ITR and packaging/enhancer region can be employed as the 5′ adenovirus sequence in the recombinant adenoviral genome. With reference to the adenovirus serotype 5 (Ad5) genome, this left terminal (5′) sequence of the Ad genome useful in this invention spans bp 1 to about 360, also referred to as map units 0-1 of the viral genome. This sequence includes the 5′ ITR, which functions as an origin of replication, and the packaging/enhancer domain, which contains sequences necessary for packaging linear Ad genomes and enhancer elements for the E1 promoter. See, e.g., P. Hearing et al, J. Virol., 61(8):2555-2558 (1987); M. Grable and P. Hearing, J. Virol., 64(5): 2047-2056 (1990); and M. Grable and P. Hearing, J. Virol., 66(2):723-731 (1992). The 3′ adenovirus sequences of the recombinant viral genome include the right terminal (3′) ITR sequence of the adenoviral genome which, with reference to Ad5, spans about bp 35,353—end of the adenovirus genome, or map units ˜98.4-100.

Preferably, the adenovirus 5′ and 3′ regions are employed in the vector in their native, unmodified form. However, some modifications including nucleotide deletions, substitutions and additions to one or both of these sequences which do not adversely effect their biological function may be acceptable. See, e.g., International Patent Publication No. WO 93/24641, published Dec. 9, 1993. The ability to modify these ITR sequences is within the ability of one of skill in the art. See, e.g., texts such as Sambrook et al, “Molecular Cloning. A Laboratory Manual.”, 2d edit., Cold Spring Harbor Laboratory, Cold Spring Harbor, N.Y. (1989).

The DNA sequences of a number of adenovirus types are available from Genbank, including type AdS [Genbank Accession No. M73260]. The adenovirus sequences may be obtained from any known adenovirus serotype, such as serotypes 2, 3, 4, 7, 12 and 40, and further including any of the presently identified human types [see, e.g., Horwitz, cited above]. Similarly adenoviruses known to infect non-human animals (e.g., chimpanzees) may also be employed in the vector constructs of this invention. See, e.g., U.S. Pat. No. 6,083,716. The selection of the adenovirus type is not anticipated to limit the following invention. A variety of adenovirus strains are available from the American Type Culture Collection, Manassas, Va., or available by request from a variety of commercial and institutional sources. Further, the sequences of many such strains are available from a variety of databases including, e.g., PubMed and GenBank. In the following examples an adenovirus type 5 (Ad5) is used for convenience. However, one of skill in the art will understand that comparable regions derived from other adenoviral strains may be readily selected and used in the present invention in the place of (or in combination with) Ad5.

In one embodiment, the adenoviral genome used in this invention contains only the minimal adenoviral sequences described above and lacks adenovirus sequences encoding functional adenoviral genes. Functional adenoviral genes include E1a, E1b, E2a, E2b, E3, E4 (or the ORF6 fragment thereof), the intermediate genes (IVa and IX) and late genes (L1, L2, L3, L4, L5). The recombinant Ad genome contains Ad 5′ and 3′ cis-elements, as well as the transgene sequences described below. Suitably, these 5′ and 3′ elements may flank the transgene (e.g., 5′ cis-elements, transgene, 3′ cis-elements). Alternatively, these 5′ ITRs and 3′ ITRs may be oriented in a head-to-tail configuration, located upstream of the transgene. Such a recombinant genome may be constructed using conventional genetic engineering techniques, e.g., homologous recombination and the like. See, e.g., U.S. Pat. No. 6,001,557.

However, in another embodiment, the recombinant adenoviral genome contains both the minimal adenoviral sequences and adenoviral sequences encoding functional adenoviral genes. Most suitably, the recombinant adenoviral genomes are deleted in E1a, E1b in order to prevent replication of the resulting viruses in the absence of complementing adenoviral E1a and E1b function. In one desirable embodiment, the recombinant adenoviral genome contains sequences encoding all adenoviral gene functions, with the exception of E1a, E1b, and E3. However, the recombinant adenoviral genomes may be deleted of any of the adenoviral gene functions, provided that the required functions for replication and packaging are provided by the packaging cell line and/or a helper. Required functional genes include E1a, E1b, E2a, E4, the intermediate genes (IVa and IX) and late genes (L1, L2, L3, L4, L5). Replacement of the adenoviral E3 gene function is not necessary. In one example below, a recombinant genome containing the adenoviral gene with a deletion in E1a, E1b, and a transgene inserted into the region deleted from the native Ad E3 region is provided.

Suitably, a plasmid according to the invention contains a rare restriction enzyme recognition site flanking the 5′ Ad ITRs and the 3′ Ad ITRs of the viral genome. Yet other plasmids of the invention contain recombinant Ad genomes from which one or more adenoviral genes selected from E1a, E1b, E2a, E3, E4 ORF6, the intermediate genes and/or the late genes, are deleted. In still another embodiment, the plasmid of the invention contains non-adenoviral genomes including, without limitation, parvoviruses, adeno-associated viruses, retroviruses, herpesviruses, and lentiviruses, among others. In such cases, one of skill in the art can readily select other suitable viral sequences for use in the present invention.

2. The Transgene

The transgene sequence contained in the recombinant viral genome (and the virus resulting from the method of the invention) is a nucleic acid sequence, heterologous to the viral sequences, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting virus will be put. For example, one type of transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, calorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, desirably, the transgene is a non-marker sequence encoding a product which is useful in biology and medicine, such as proteins, peptides, anti-sense nucleic acids (e.g., RNAs), enzymes, or catalytic RNAs. The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in a host cell. The invention further includes using multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M L. Donnelly, et al, J. Gen. Virol., 78(Pt 1) 13-21 (Jan 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. However, the selected transgene may encode any product desirable for study. The selection of the transgene sequence is not a limitation of this invention.

Useful products encoded by the transgene include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-18, monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and M[HC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP 1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a gene. Other suitable transgenes may be readily selected by one of skill in the art. The selection of the transgene is not considered to be a limitation of this invention.

3. Regulatory Elements

In addition to the major elements identified above for the viral vector, the plasmid and/or recombinant viral genome also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1a promoter [Invitrogen]. Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a transgene operably linked to a tissue-specific promoter. For instance, if expression in skeletal muscle is desired, a promoter active in muscle should be used. These include the promoters from genes encoding skeletal a-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al, J. Bone Miner. Res., 11:654-64 (1996)), lymphocytes (CD2, Hansal et al., J. Immunol., 161:1063-8 (1998); immunoglobulin heavy chain; T cell receptor a chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88 5611-5 (1991)), and the neuron-specific vgf gene (Piccioli et al., Neuron, 15:373-84 (1995)), among others.

Optionally, plasmids carrying therapeutically useful transgenes may also include selectable markers or reporter genes may include sequences encoding geneticin, hygromicin or purimycin resistance, among others. Such selectable reporters or marker genes (preferably located outside the viral genome to be rescued by the method of the invention) can be used to signal the presence of the plasmids in bacterial cells, such as ampicillin resistance. Other components of the plasmid may include an origin of replication. Selection of these and other promoters and vector elements are conventional and many such sequences are available [see, e.g., Sambrook et al, and references cited therein].

The combination of the transgene, promoter/enhancer, and the other regulatory vector elements is referred to as a “minigene” for ease of reference herein. In one embodiment, the minigene is located between the 5′ and 3′ cis-acting adenovirus sequences described above and, optionally, is inserted into the region of deleted adenoviral sequences (e.g., in the E1 or E3 site). Such a minigene may have a size in the range of several hundred base pairs up to about 30 kb depending upon the number of adenovirus early and late gene sequences which have been deleted from the recombinant adenoviral genome. Provided with the teachings of this invention, the design of such a minigene can be made by resort to conventional techniques.

4. Plasmid Backbones

As described herein, the invention provides an uncut, circular plasmid carrying, at a minimum, a recombinant viral genome as described herein, in which the viral genome is flanked at its 5′ and 3′ ends by one or more rare restriction enzyme sites.

Most suitably, the plasmid backbone is a plasmid which replicates in low copy number in the host cell (e.g., about 10 to 20 copies per host cell), thereby avoiding instability of the host cell. Desirably, the plasmid may be derived from any suitable source, including, without limitation, bacterial sources; insect, e.g., baculovirus expression; or yeast, fungal, or viral sources. The plasmid is relatively small, thereby permitting accommodation of the relatively large adenoviral genome and any additional, optional expression cassette. Other appropriate circular DNA vectors, of which numerous types are known in the art, can also be used for this purpose. Methods for obtaining such circular DNA plasmid vectors are well-known. See, e.g., Promega Protocols and Applications Guide (3d ed. 1996), eds. Doyle, ISBN No. 1-882274-57-1; Sambrook et al, Molecular Cloning. A Laboratory Manual, 2d edition, Cold Spring Harbor Laboratory, New York (1989); Miller et al, Genetic Engineering, 8:277-298 (Plenum Press 1986) and references cited therein.

Suitable plasmid (DNA) backbones have been described in the literature [e g., pAdLink, which contains native Ad5 mu 0-1, a polycloning site, and Ad5 m.u. 9-16; Gil Hong Parl et al, Korean J. Biochem., 27:91-97 (1995); and d11004, which is an Ad5 mutant viral backbone with a 1.9 kb deletion in the E4 region, Bridge and G. Katner, J. Virol., 63:6031-6038 (1989)]]. These and other plasmid backbones may be obtained from a variety of academic and commercial sources. See, e.g., American Type Culture Collection, 10801 University Boulevard, Manassas, Va. 20110-2209; Clontech (Palo Alto, Calif.); Stratagene (La Jolla, Calif.), among others.

The plasmids of the invention may be generated by resort to the techniques reference herein, as well as a variety of other techniques which are well known to those of skill in the art. Once assembled, the plasmid may be optionally subject to purification.

Preferably, the plasmid carrying a viral genome for rescue and packaging, which is flanked by rare restriction enzyme sites, is transfected into the host cell, where it may exist transiently or preferably as an episome until the rare restriction enzyme cleaves its corresponding sites, thereby rescuing the viral genome. In the case of a recombinant adenoviral genome, the rare restriction enzyme sites are located 5′ to the 5′ inverted terminal repeat sequences (ITRs) and 3′ to the 3′ ITRs.

III. Providing Rare-Cutting Restriction Enzymes to Host Cells

The rare-cutting restriction enzyme selected for use in the present invention may be delivered to the cell in trans, or expressed from a host cell stably transformed with a molecule encoding the enzyme. When expressed in trans, the rare-cutting restriction enzyme may be expressed from a separate molecule delivered to the host cell, or, alternatively, the rare restriction enzyme may be expressed from an expression cassette which is carried on the same plasmid which carries the adenoviral genome.

A. The Molecule Encoding the Rare-Cutting Restriction Enzyme

Thus, sequences encoding the rare-cutting restriction enzyme, e.g., I-SceI, or a functional fragment thereof may be provided to a selected host cell in trans. The nucleic acid molecule carrying the rare-cutting restriction enzyme (e.g, I-SceI) coding sequences and expression control sequences may be in any form which transfers these components to the host cell and permits expression of the restriction enzyme, preferably in the cell nucleus. These sequences may be provided as “naked DNA”. Most suitably, however, these sequences are contained within a vector. A “vector” includes, without limitation, any genetic element, such as a plasmid, phage, transposon, cosmid, chromosome, virus, virion, etc.

The selected delivery vector contains the restriction enzyme nucleic acid sequences and regulatory elements which permit transcription, translation and/or expression of the enzyme in a host cell containing the enzyme delivery vector. Desirably, the nucleic acid molecule encoding the rare-cutting restriction enzyme, e.g., I-SceI, is further provided with a nuclear localization signal, which targets the I-SceI sequences to the nucleus. Suitable nuclear localization signals are known to those of skill in the art and are not a limitation of the present invention.

Other expression control elements include promoters, including both constitutive and inducible promoters, as are described above, poly A sequences, and the like. Other heterologous nucleic acid sequences optionally present in this enzyme delivery virus include sequences providing signals required for efficient polyadenylation of the RNA transcript, and introns with functional splice donor and acceptor sites. A common poly-A sequence which is employed in the enzyme delivery viruses useful in this invention is that derived from the papovavirus SV-40. For example, in a rAAV delivery virus, the poly-A sequence generally is inserted following the sequences for rare restriction enzyme and before the 3′ AAV ITR sequence. An enzyme delivery vector useful in the present invention may also contain an intron, desirably located between the promoter/enhancer sequence and the transgene. One possible intron sequence is also derived from SV-40, and is referred to as the SV-40 T intron sequence. These and other common vector elements are discussed herein and may be readily selected by one of skill in the art [see, e.g., Sambrook et al, and references cited therein at, for example, pages 3.18-3.26 and 16.17-16.27]. Optionally, the enzyme delivery vector may contain a selectable marker or reporter sequences, such as sequences encoding hygromycin or purimycin, among others. See the discussion of reporter sequences herein.

In one embodiment, the restriction enzyme coding sequences are delivered via a viral vector, and most preferably, via a recombinant, infectious virus. Selection of the enzyme delivery virus is not a limitation on the present invention. Suitable recombinant enzyme (e.g., I-SceI) delivery viruses may be readily engineered utilizing such viruses as adeno-associated viruses (AAV), retroviruses, adenoviruses, hybrid adeno-AAV viruses, lentiviruses, baculovirus, herpes virus, and pox viruses, among others.

The vectors carrying the rare restriction enzyme of the invention are prepared using the rare-cutting restriction enzyme sequences, obtained as described herein, and using known methods. For example, methods for producing rAAV vectors have been described. [See, W. Xiao et al, J. Virol., 72:10222-10226 (1998); U.S. Pat. No. 5,658,776; U.S. Pat. No. 5,622,856, among others].

In another embodiment, the sequence encoding the selected rare restriction enzyme (e.g., I-SceI) may be engineered into the plasmid which contains the recombinant Ad genome flanked by rare restriction enzyme sites, e.g., to provide a bicistronic plasmid. The restriction enzyme may be expressed either under the control of a constitutive promoter or a regulatable promoter which expresses the enzyme following activation. These and other regulatory control elements which may be located on such a bicistronic plasmid are discussed elsewhere in the specification. The engineering methods used to construct any embodiment of this invention are known to those with skill in nucleic acid manipulation and include genetic engineering, recombinant engineering, and synthetic techniques. See, e.g., Sambrook et al, cited above; and International Patent Application NO. WO95/13598.

Optionally, expression of the restriction enzyme may be terminated by “shutting off” a regulatable promoter. Alternatively, or additionally, it may be desirable to de-activate expressed restriction enzyme by environmental means. For example, the activity of the I-SceI enzyme is thermally unstable and may be destroyed by incubating the host cell at a temperature (e.g., about 39° C.) which destroys further I-SceI activity without affecting the rescue of the recombinant adenoviral genome. Desirably, these or other environmental means for deactivating the selected rare-cutting restriction enzyme are employed following cleavage of the rare restriction enzyme sites by the corresponding enzyme. Optionally, the activity of a restriction enzyme may be de-activated by heat or other environmental means, irrespective of whether the rare restriction enzyme is provided in trans, from the circular plasmid containing the adenoviral genome, from another DNA molecule, or from the cell itself In another embodiment, the sequences encoding the rare restriction enzyme are provided with a self-termination element. This is particularly well suited for use when the sequences are contained on the same plasmid as the Ad genome. For example, the circular plasmid may contain the adenoviral genome flanked by rare restriction enzyme sites, and the sequence encoding the rare restriction enzyme may also be flanked by or contain rare restriction enzyme sites so that following expression, the enzyme cleaves the sites flanking the adenoviral genome, thereby rescuing the adenoviral genome. Further, the rare restriction enzyme sites are engineered into the sequence encoding the rare restriction enzyme so that cleavage prevents further expression of the enzyme. This can be accomplished by inserting the rare restriction enzyme sites between the coding sequence for the enzyme and its expression control sequences.

The nucleic acid molecule selected to carry the sequences encoding the rare-cutting restriction enzyme (e.g., I-SceI), as well as the sequences which regulate expression thereof, may be provided to the host cell by any suitable method. Examples of suitable methods are described in more detail below.

V. Host Cells

The invention provides host cells which are useful in the production of viral vectors. The host cell itself may be selected from any biological organism, including prokaryotic (e.g., bacterial) cells, and eukaryotic cells, including, insect cells, yeast cells and mammalian cells.

Most preferably, the cells used for transfection and production of infectious virus are from a mammalian cell line, including, without limitation, cells such as A549, WEHI, the murine 3T3 cells derived from Swiss, Balb-c or NIH mice, 10T1/2, BHK, MDCK, COS1, COS7, BSC 1, BSC 40, BMT 10, VERO, W138, HeLa, Saos, C2C12, L cells, HT1080, HepG2, CV-1, and primary fibroblast, hepatocyte and myoblast cells derived from mammals including human, monkey, mouse, rat and hamster. Preferred cells include human cells, and most preferably, cells which express adenovirus El functions, e.g., 293 cells. Still other suitable mammalian host cells, as well as methods for transfection, culture, amplification, screening, production, and purification are known in the art. [See, e.g., Gething and Sambrook, Nature, 293:620-625 (1981), or alternatively, Kaufman et al, Mol. Cell. Biol., 5(7):1750-1759 (1985) or Howley et al, U.S. Pat. No. 4,419,446].

In one desirable embodiment in which the plasmid carries an adenoviral genome, the cell line selected is stably transformed with the E1a and E1b gene functions, which permit replication and packaging of an E1-deleted, replication-defective, adenoviral genome into an infectious particle. One suitable cell line for this use is the human 293 cell line, or cell lines derived therefrom to express other desired adenoviral functions, or to express a desired rare restriction enzyme.

Thus, in one embodiment, the invention provides cell lines which stably express a rare-cutting restriction enzyme. For convenience throughout this specification, reference will be made to the I-SceI enzyme. However, it will be readily understood that another rare-cutting restriction enzyme and/or its restriction enzyme site, as defined herein, may be substituted.

A. Stable Cell Line Expressing Rare-Cutting Restriction Enzyme Functions

A cell line of the invention may be constructed by providing the selected host cell line with a nucleic acid molecule encoding a rare-cutting restriction enzyme or a functional fragment thereof operably linked to sequences which control expression thereof using conventional techniques. Such expression control sequences are described above. As used herein, a “functional fragment” is a portion of the coding sequence or enzyme which performs the same or substantially the same function as the full coding sequence (e.g., encoding a functional enzyme or fragment thereof) or enzyme (e.g., a portion of the enzyme which cleaves at the site recognized by the full-length enzyme).

In one embodiment, the host cell stably expresses the rare-cutting restriction enzyme under the control of a constitutive promoter, examples of which are provided herein. In another embodiment, the rare-cutting restriction enzyme is stably expressed by the host cell under the control of an inducible promoter, examples of which are provided herein.

B. Other Functions Provided by Host Cell

The host cell is stably transformed, or expresses, any sequences necessary for packaging the viral genome into a capsid or envelope protein. In the case of a plasmid carrying an adenoviral genome deleted of adenoviral gene functions, these sequences include early genes E1, E2, E4 ORF6, or fragments of a gene which perform the same or substantially the same function as the intact complete gene (i.e., functional fragments), and all remaining late, intermediate, structural and non-structural genes of the adenovirus genome, which are not present in the pAd or provided by the cell line.

Most suitably, the helper virus and cell line provide, at a minimum, adenovirus E1a and E1b functions. However, where necessary, the helper virus and cell line provides one or more of the necessary gene functions selected from among E1a, E1b, E2a, E4, and/or VAI, or functional fragments of these genes (e.g., E4 ORF6) and the adenoviral gene IX function. Preferably, the resulting recombinant virus is an adenovirus and, most preferably, an adenovirus which replicates in the presence of the gene functions provided in the selected host cell. Alternatively, other suitable viruses, e.g., herpesviruses, may be used as helpers.

VI. Production of Recombinant Virus

The compositions and methods of the present invention are particularly useful for rescue of a recombinant viral genome during production and for packaging of the genome into a viral capsid or envelope. Together, the host cell and the plasmid carrying the viral genome (and optionally a helper) provide the gene functions necessary to package the recombinant virus. This present invention may be utilized for any adenoviral vector, whether helper-dependent or independent for packaging. In addition, this method may be used for production of any other viral vectors including, without limitation, alphavirus, adenovirus, baculoviruses, delta virus, hepatitis viruses, herpes viruses, lentiviruses, filoviruses, pox viruses, papova viruses, poliovirus, pseudorabies viruses, parvoviruses (e.g., recombinant adeno-associated vectors (rAAV)), retroviruses, and vaccinia viruses, amongst others parvoviruses (e.g., recombinant adeno-associated vectors (rAAV)), hybrid adenovirus/AAV vectors, among others. Selection of a suitable viral vector is not a limitation of the present invention.

In the performance of the method of the invention, the circular DNA construct (i.e., plasmid) carrying the recombinant viral genome flanked by rare-cutting restriction enzyme recognition sites is delivered to the host cells using conventional techniques. Advantageously, the method of the invention avoids the need to linearize the plasmid prior to transfection of a host cell, as described in prior art methods such as Fisher et al, Virol., 217:11-22 (1996), which is incorporated by reference herein. The transfection may then performed using, e.g., the calcium-phosphate based techniques described in Cullen, in “Methods in Enzymology”, ed. S. L. Berger and A. R. Kimmel, Vol. 152, pp. 684-704, Academic Press, San Diego (1987). Other suitable transfection techniques are known and may readily be utilized to deliver the recombinant vector to the host cell. Alternatively, delivery can be by another suitable method, such as is described herein.

Generally, when delivering the recombinant vector (e.g., pAd) by transfection, the vector is delivered in an amount from about 1 μg to about 100 μg DNA, and preferably about 5 μg to about 50 μg DNA to about 1×10⁴ cells to about 1×10¹³ cells. More preferably, about 10 μg to about 25 μg DNA is delivered to about 1×10⁵ to about 1×10⁷ cells, and most preferably about 5 μg DNA is delivered to about 5×10⁶ cells. However, the relative amounts of vector DNA to host cells may be adjusted, taking into consideration such factors as the selected vector, the delivery method and the host cells selected.

In one embodiment, the plasmid of the invention is transfected into a host cell which stably expresses the selected rare-cutting restriction enzyme (e.g., I-SceI). In an alternative embodiment, the host cell is transiently transfected or infected with the sequences encoding the rare-cutting restriction enzyme (e.g., I-SceI). The plasmids and, optionally, any separate nucleic acid molecule carrying the sequences encoding the rare-cutting restriction enzyme (e.g., I-SceI) and its regulatory control sequences, are provided to the host cell by any suitable method. Such suitable methods may include transfection, electroporation, liposome delivery, membrane fusion techniques, high velocity DNA-coated pellets, viral infection and protoplast fusion. See, for instance, Sambrook et al, Molecular Cloning: A Laboratory Manual, Cold Spring Harbor Laboratories, Cold Spring Harbor, N.Y.

Suitably, the rare restriction enzyme delivery virus may be delivered to the selected host cells at a multiplicity of infection (MOI) of about 5 to about 200 genome particles (e g., rAAV), and preferably at an MOI of 10 to 100 genome particles Suitable MOI for other selected enzyme delivery viruses may be in this range, or may be adjusted as desired by one of skill in the art. Alternatively, where the I-SceI is delivered by a vector which lacks the ability to infect host cells, the vector is delivered to the host cells in an amount of about 5 μg to about 100 μg DNA by any suitable means known to those of skill in the art.

The enzyme delivery vehicle may be provided to the host cells at any time prior to cell lysis, including prior to delivery of the plasmids, prior to delivery of the helper virus sequences (if any), or after delivery of either or both of these components to the host cell. For example, where the enzyme delivery vehicle constitutively expresses the restriction enzyme (e.g., I-SceI), it may be desirable to provide this enzyme delivery vehicle to the host cells following delivery of the plasmids and the helper virus. Alternatively, where the enzyme delivery vehicle inducibly expresses the restriction enzyme, the timing of delivery of the enzyme may not be critical. However, the selection of promoters, and the determination of timing of delivery of an enzyme delivery virus (or other nucleic acid molecule) to the host cell may be made by one of skill in the art in view of the information provided herein.

The host cell, provided with the recombinant plasmid carrying the viral genome and any optional helper virus as described above, is then cultured in a similar manner to provide recombinant virus in a viral capsid (e.g., adenovirus) or envelope. Optionally, the coding sequences for the rare restriction enzyme may be carried on the same plasmid carrying the viral genome. See, generally, Sambrook et al, cited above. See also, the methods described in K. J. Fisher et al, Virol., 217:11-22 (1996).

In one embodiment, the host cell may be subjected to conditions which de-activate the rare restriction enzyme after it has had sufficient time to rescue the viral genome (i.e., cut at the rare restriction enzyme recognition sites). For example, the host cell may be incubated at a temperature (e.g., about 39° C.) which destroys further enzyme activity without degrading the DNA. However, alternative means for deactivating the rare restriction enzyme may be readily selected by one of skill in the art and is not a limitation of the present invention.

Suitably, the host cell containing the plasmids carrying the viral genome, the rare restriction enzyme, and the optional helper virus, is cultured under suitable conditions to permit production of recombinant viral vector in a first round of amplification. Regardless of which production method is utilized, the recombinant viruses produced according to the invention may be readily purified from culture using methods known to those of skill in the art. One suitable method involves ultracentrifugation with or without sucrose or affinity chromatography. Conventional techniques may be used to concentrate the recombinant virus (see, e.g., J. C. Burn et al, Proc. Natl. Acad. Sci. USA, 90:8033-8037 (1993)).

In one embodiment, recombinant viruses resulting from the method of the invention are suitable for applications in which transient transgene expression or delivery of another selected molecule is therapeutic. However, the recombinant viruses are not limited to use where transient transgene expression is desired. The recombinant viruses are useful for a variety of situations in which delivery and expression of a selected molecule is desired including, functional genomic and other research purposes, as well as therapeutic, and vaccine purposes, among others, which are well known to those of skill in the art.

VII. Pharmaceutical Compositions

The recombinant viruses according to the present invention are suitable for a variety of uses including in vitro protein and peptide expression, as well as ex vivo and in vivo gene delivery.

The recombinant viruses produced according to the invention may be used to deliver a selected molecule to a host cell by any suitable means. In one embodiment, the transfer viruses and the cells are mixed ex vivo; the infected cells are cultured using conventional methodologies; and the transduced cells are re-infused into the patient.

Alternatively, the recombinant viruses, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the recombinant viruses and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

VIII. Gene Delivery Methods

Thus, the invention provides a method of delivering a transgene or other molecule to a human or veterinary patient by transducing the cells of the patient with a recombinant virus produced according to the invention. The target cells may be transduced in vivo or in vitro, taking into consideration such factors as the selection of target cells, the transgene being delivered, and the condition for which the patient is being treated.

A. In vivo

For in vivo delivery of the transgenes, any suitable route of administration may be used, including, direct delivery to the target organ, tissue or site, intranasal, intravenous, intramuscular, subcutaneous, intradermal, vaginal, rectal, and oral administration. Routes of administration may be combined within the course of repeated therapy or immunization.

Suitable doses of viruses may be readily determined by one of skill in the art, depending upon the condition being treated, the health, age and weight of the veterinary or human patient, and other related factors. However, generally, a suitable dose may be in the range of 10³ to 10¹⁸ , preferably about 10⁵ to 10¹⁶ plaque forming units (PFU) per dose, and most preferably, about 10⁷ to 10⁹ PFU for an adult human having a weight of about 80 kg. This dose may be formulated in a pharmaceutical composition, as described above (e.g., suspended in about 0.01 mL to about 1 mL of a physiologically compatible carrier) and delivered by any suitable means. The dose may be repeated, as needed or desired, daily, weekly, monthly, or at other selected intervals.

B In Vitro

In another embodiment, the viruses of the invention are useful for in vitro transduction of target cells. Optionally, the viruses may be used in ex vivo therapy, which involves removal of a population of cells containing the target cells, transduction of the cells in vitro, and then reinfusion of the transduced cells into the human or veterinary patient.

Optionally, the host cells which have been transfected or infected in vitro with the constructs described above may themselves be administered to a host. These cells are desirable cells of the same species as the species of the host into which they are intended to be delivered, e.g., mammalian cells, such as C127, 3T3, CHO, human kidney 293, are useful host cells. These cells can be made using techniques known in the art. Preferably, the cells may be harvested from the specific host to be treated and made into donor cells by ex vivo manipulation, akin to adoptive transfer techniques, such as those described in D. B. Kohn et al, Nature Med., 4(7):775-80 (1998).

Generally, when used for in vitro transduction or ex vivo therapy, the targeted host cells are infected with 10⁵ plaque forming units (PFU) or genome copies (GC) to 10¹⁰ PFU (genome copies) viruses for each 10¹ to 10¹⁰ cells in a population of target cells. However, other suitable dosing levels may be readily selected by one of skill in the art.

The following examples are provided to illustrate methods for producing the compositions useful in the method of the invention and methods for performing the invention. Such examples do not limit the scope of the present invention. One skilled in the art will appreciate that although specific reagents and conditions are outlined in the following examples, modifications can be made which are meant to be encompassed by the spirit and scope of the invention.

### EXAMPLE 1

### Rescue of Adenovirus Following Transient Transfection of E1-Expressing Cells With Plasmid I-SceI Enzyme

A plasmid used in the following experiment carried a recombinant adenoviral genome containing the entire adenovirus genome with deletions in the E1a and E1b and E3 regions, with a cassette comprising the green fluorescent protein (GFP) expressed under the cytomegalovirus (CMV) promoter inserted in the site of the E1 deletion. The plasmid further contains PacI sites and I-SceI sites. The PacI sites are engineered to be located immediately adjacent to the I-SceI sites. The plasmid was further designed to contain a single I-SceI site 5′ to the 5′ end of the Ad genome (i.e., the Ad 5′ inverted terminal repeat, ITR) and a single I-SceI site 3′ to the 3′ end of the Ad genome (i.e., the Ad 3′ ITR). The I-SceI sites are unique in the adenoviral genome and the remainder of the plasmid. This plasmid is termed pAdCMVGFP.

The following manipulations were performed in this study. The circular pAdCMVGFP with PacI and I-SceI sites flanking the ITRs was transfected into 293 cells in 6 well plates containing about 1×10⁶ cells. The transfected cells were top-agar overlaid at 24 hours post transfection and green plaques were counted under a UV-microscope at day 14 post transfection. (Table I(a)).

pAdCMVGFP was linearized by digestion with I-SceI, releasing the recombinant adenoviral genome, prior to transfection in 293 cells as described above. See, Table I(b).

pAdCMVGFP was linearized by digestion with PacI prior to transfection into 293 cells as described above. See, Table I(c).

The circular pAdCMVGFP with PacI and I-SceI sites flanking the ITRs was transfected into 293 cells. A plasmid expressing the I-SceI endonuclease from the rous sarcoma virus (RSV) promoter was co-transfected with the 293 cells as described above. This plasmid, pRSV-I-SceI, was obtained by cloning the digested and excised I-SceI into pRep7 [Invitrogen] after the RSV promoter. See, Table I(d).

The results of these manipulations are provided in Table I below.

TABLE I

| | No. of plagues |
|---|---|
| (a) 2 μgs of pAdCMVGFP with Pac I and I-Sce I sites | 0 |
| flanking ITRs | |
| (b) 2 μgs of pAdCMVGFP with Pac I and I-Sce I sites | 34 |
| flanking ITRs digested with I-Sce I before transfection | |
| (c) 2 μgs of pAdCMVGFP with Pac I and I-Sce I sites | 10 |
| flanking ITRs digested with Pac I before transfection | |
| (d) 2 μgs of pAdCMVGFP with Pac I and I-Sce I sites | 49 |
| flanking ITRs and 1 μg of pRSV-I-Sce I | |

These data demonstrate that co-transfection of uncut pAd plasmid with I-Sce I expression plasmid resulted in more efficient rescue of adenovirus (d) than that of transfection of linearized adenovirus plasmids (b) and (c). In addition, I-Sce I expressed in 293 cells can specifically recognize implanted I-Sce I sites on the adenovirus plasmid and cleave them efficiently (d).

### EXAMPLE 2

### Rescue of Adenovirus Following Transfection of Stable Cell Line Expressing Cells Adenovirus E1 and I-SceI Enzyme

A cell line derived from 293 cells which is stably transformed with I-SceI endonuclease and constitutively expresses the endonuclease from the elongation factor promoter was used in this experiment. This cell line has been described in WO 00/75353, published Dec. 14, 2000.

Cells from the stable 293/1-SceI cell lines were transfected with either pAdCMVGFP which has been linearized by digestion with PacI (cut) or which remained circular (uncut) in the amounts shown in Table II below.

TABLE II

| | No. Green Plagues | | | |
|---|---|---|---|---|
| Transfection/293-I-SceI cells | Day 5 | Day 6 | Day 7 | Day 8 |
| 0.5 μg | | | | |
| cut | 0 | 0 | 0 | 1 |
| uncut | 1 | 2 | 4 | 13 |
| 1 μg | | | | |
| cut | 0 | 2 | 3 | 5 |
| uncut | 10 | 35 | 72 | 140 |
| 2 μg | | | | |
| cut | 0 | 8 | 12 | 14 |
| uncut | 10 | 76 | 140 | >280 |

These circular adenovirus plasmid rescue experiments demonstrate that plaque efficiency was enhanced 10-30 fold in the stable 293/1-SceI cell line, as compared with that of the linearized (cut) plasmid transfected into the same cell line.

### EXAMPLE 3

### Cellular Concentrations of Circular Adenovirus Plasmid Required for Efficient Rescue

A stable 293-1-SceI cell line was transfected with 0.5 μg, 1 μg, 2 μg, or 4 μg of pAdCMVGFP, as described above, and green plaques were counted on day 8 through day 12 post-transfection.

The data (not shown) demonstrates that cellular concentrations of circular adenovirus genomes are critical to efficient virus rescue in 293-1-SceI cells. While rescue was possible following transfection with 1 μg circular plasmid DNA, more efficient rescue was achieved when transfection concentrations exceeded 2 μg plasmid DNA. Thus, based on the size of the plasmid and the type of cell culture utilized, a concentration of 2 to 4 μg appeared to provide the best results.

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims.

What is claimed is:

1. A method of rescuing a recombinant viral genome, said method comprising the steps of
(a) transfecting into a host cell an uncut, circular plasmid containing a recombinant viral genome, a first rare restriction enzyme recognition site located 5′ to the viral genome, and a second rare restriction enzyme recognition site located 3′ to the viral genome;
(b) expressing in the host cell at least one restriction enzyme which specifically recognizes the first and second rare restriction enzyme recognition sites; and
(c) culturing the host cells under conditions in which the rare restriction enzyme cleaves the rare restriction enzyme recognition sites, thereby rescuing the recombinant viral genome from the plasmid.

2. The method according to claim 1, wherein the rare restriction enzyme recognition sites are located immediately 5′ and/or immediately 3′ to the viral genome.

3. The method according to claim 1, wherein the plasmid comprises a spacer of about 1 to about 10 nucleotides between the 5′ end of the viral genome and the first rare restriction enzyme recognition site and/or between the 3′ end of the viral genome and the second rare restriction enzyme recognition site.

4. The method according to claim 1, wherein the spacer comprises a further restriction enzyme site unique to the plasmid.

5. The method according to claim 1, wherein the plasmid comprises at least one further rare restriction enzyme recognition site located adjacent to the first rare restriction enzyme recognition site.

6. The method according to claim 1, wherein the plasmid comprises at least one further rare restriction enzyme recognition site located adjacent to the second rare restriction enzyme recognition site.

7 The method according to claim 1, wherein the uncut, circular plasmid further contains a nucleic acid sequence encoding the rare restriction enzyme under the control of sequences which regulate its expression.

8. The method according to claim 7, wherein a first rare restriction enzyme site is located 5′ to the nucleic acid sequence encoding the rare restriction enzyme and a second rare restriction enzyme site is located 3′ to the regulatory control sequences for the enzyme.

9. The method according to claim 7, wherein a rare restriction enzyme site is located between the sequence encoding the rare restriction enzyme and the sequences which regulate its expression.

10. The method according to claim 1, further comprising the step of deactivating the rare restriction enzyme following cleavage of the rare restriction enzyme site.

11. The method according to claim 1, wherein the plasmid is a bacterial plasmid.

12. The method according to claim 1, wherein the rare restriction enzyme is stably integrated into the host cell.

13. The method according to claim 12, wherein the rare restriction enzyme is expressed under the control of a regulatable promoter.

14. The method according to claim 13, wherein the rare restriction enzyme is regulated by a molecule provided on the transfected plasmid.

15. The method according to claim 1, wherein the rare restriction enzyme is expressed under the control of a constitutive promoter.

16. The method according to claim 1, wherein the rare restriction enzyme is provided in trans.

17. The method according to claim 16, wherein the rare restriction enzyme is provided by transfection of the host cell.

18. The method according to claim 16, wherein the rare restriction enzyme is provided by infection of the host cell.

19. The method according to claim 1, wherein the recombinant viral genome comprises adenoviral 5′ inverted terminal repeat sequences (ITRs) and adenoviral 3′ ITRs.

20. The method according to claim 19, wherein the recombinant viral genome further comprises a transgene.

21. The method according to claim 19, wherein the recombinant viral genome is an adenovirus which lacks the ability to express functional E1a and/or E1b proteins.

22. The method according to claim 21, wherein the recombinant adenoviral genome comprises 5′ ITRs, adenoviral sequences encoding E2a, a transgene, and adenoviral sequences encoding E4 ORF6.

23. The method according to claim 1, wherein one or more of the rare restriction enzyme recognition sites is a I-SceI recognition site and the rare restriction enzyme is I-SceI endonuclease.

24. The method according to claim 1, wherein one or more of the rare restriction enzymes is independently selected from the group consisting of PspI and I-CeuI.

25. A host cell containing:
(a) an uncut, circular plasmid containing a recombinant adenovirus genome, a first rare restriction enzyme recognition site located 5′ to the adenovirus genome, and a second rare restriction enzyme recognition site located 3′ to the adenovirus genome; and
(b) nucleic acid sequences encoding at least one restriction enzyme which specifically recognizes the first and second rare restriction enzyme recognition sites.

26. The host cell according to claim 25, wherein the host cell is stably transformed with a molecule comprising the nucleic acid sequences which permit expression of the rare restriction enzyme in the host cell.

27. The host cell according to claim 25, wherein the nucleic acid sequences encoding the rare restriction enzyme are operably linked to a regulatable promoter which directs expression thereof.

28. The host cell according to claim 25, wherein the nucleic acid sequences encoding the rare restriction enzyme are operably linked to a constitutive promoter which directs expression thereof.

29. The host cell according to claim 25, wherein the nucleic acid sequences encoding the rare restriction enzyme and regulatory sequences which direct expression thereof are contained on the uncut, circular plasmids.

30. The host cell according to claim 29, wherein a third rare restriction enzyme site is located 5′ to the nucleic acid sequences encoding the rare restriction enzyme and regulatory sequences which direct expression thereof and a fourth rare restriction enzyme site is located 3′ to the regulatory sequences therefor.

31. The host cell according to claim 29, wherein a rare restriction enzyme site is located between the sequence encoding the rare restriction enzyme and the sequences which regulate its expression.

32. The host cell according to claim 29, where in the host cell further comprises adenovirus sequences encoding adenovirus E1a and E1b.

33. The host cell according to claim 29, wherein one or more of the first, second, third and fourth rare restriction enzymes is I-SceI.

34. The host cell according to claim 29, wherein one or more of the first, second, third and fourth rare restriction enzymes is independently selected from the group consisting of PspI and I-CeuI

35. A method of producing a recombinant adenovirus comprising the steps of:
(a) providing a host cell comprising an uncut, circular plasmid containing a recombinant adenoviral genome, a first rare restriction enzyme recognition site located 5′ to the adenoviral genome, and a second rare restriction enzyme recognition site located 3′ to the adenoviral genome;
(b) expressing at least one rare restriction enzyme specific for the first and second rare restriction enzyme recognition sites in the host cell under conditions in which the rare restriction enzyme cleaves the rare restriction enzyme recognition sites, thereby rescuing the recombinant adenoviral genome from the plasmids; and
(d) culturing the host cell in the presence of sufficient adenoviral functions to permit encapsidation of the rescued adenoviral genome into an infectious recombinant adenovirus.

36. The method according to claim 35, further comprising the step of purifying the recombinant adenovirus from the culture.
